(19) United States
(12) Patent Application Publication
(10) Pub. No.: US 2020/0164369 A1
(43) Pub. Date: May 28, 2020

(54) MICRO-FLUIDIC CHIP, METHOD FOR SEPARATING CELLS TO BE CULTURED, AND METHOD FOR MANUFACTURING MICRO-FLUIDIC CHIP

(71) Applicants: BEIJING BOE TECHNOLOGY DEVELOPMENT CO., LTD., Beijing (CN); BOE TECHNOLOGY GROUP CO., LTD., Beijing (CN)

(72) Inventor: Yingyi LI, Beijing (CN)

(21) Appl. No.: 16/436,511

(22) Filed: Jun. 10, 2019

(30) Foreign Application Priority Data
Nov. 23, 2018 (CN) 201811409487.3

(51) Int. Cl. B01L 3/00; C12N 5/0735

(52) U.S. Cl. CPC B01L 3/502715; C12N 5/0606; B01L 2300/087; B01L 3/502746; B01L 2300/048; B01L 3/502723

(57) ABSTRACT

A micro-fluidic chip, a method for separating cells to be cultured, and a method for manufacturing the micro-fluidic chip are disclosed. The micro-fluidic chip includes a body, a culture chamber, a first channel and a second channel, the culture chamber, the first channel and the second channel are located in the body. The first channel and the second channel are intersected to form an intersection; the first channel is configured to transport a suspension liquid containing cells to be cultured; and an end of the second channel is communicated with the culture chamber and configured to inject a culture fluid into the culture chamber so as to bring a cell to be cultured disposed at the intersection into the culture chamber.

The present application claims priority of China Patent application No. 201811409487.3 filed on Nov. 23, 2018, the content of which is incorporated in its entirety as a portion of the present application by reference herein.

### TECHNICAL FIELD

Embodiments of the present disclosure relate to a micro-fluidic chip, a method for separating cells to be cultured, and a method for manufacturing the micro-fluidic chip.

### BACKGROUND

Stein cells are pluripotent cells with self-replication ability. Under certain conditions, stem cells can be differentiated into multiple functional cells. The cultivation and separation of the stem cells is of great significance in cell healing and cell regeneration.

### SUMMARY

At least one embodiment of the present disclosure provides a micro-fluidic chip, including a body and a culture chamber, a first channel and a second channel, the culture chamber, the first channel, and the second channel being located in the body, the first channel and the second channel are intersected to form an intersection; the first channel is configured to transport a suspension liquid containing cells to be cultured; and an end of the second channel is communicated with the culture chamber and configured to inject a culture fluid into the culture chamber so as to bring a cell to be cultured disposed at the intersection into the culture chamber.

For example, in the micro-fluidic chip provided by an embodiment of the present disclosure, a cross-sectional dimension of the first channel and the second channel is configured to only allow a single one of the cells to be cultured to pass through; and the second channel is configured to inject the culture fluid into the culture chamber so as to bring a single cell to be cultured disposed at the intersection into the culture chamber.

For example, in the micro-fluidic chip provided by an embodiment of the present disclosure, the cells to be cultured are stem cells.

For example, in the micro-fluidic chip provided by an embodiment of the present disclosure, an end of the second channel away from the culture chamber, the intersection, and the end of the second channel communicated with the culture chamber are arranged in a same straight line.

For example, in the micro-fluidic chip provided by an embodiment of the present disclosure, the first channel includes a first straight line part; the first straight line part and the second channel are intersected to form the intersection; and an included angle between the first straight line part and the second channel is 75°-90°.

For example, in the micro-fluidic chip provided by an embodiment of the present disclosure, a reversing valve is disposed at the intersection and provided with a valve cavity for accommodating a single one of the cells to be cultured, and has two working states: in a first working state, the first channel is switched on and the second channel is switched off; and in a second working state, the second channel is switched on and the first channel is switched off.

For example, in the micro-fluidic chip provided by an embodiment of the present disclosure, the cells to be cultured are stem cells.

For example, in the micro-fluidic chip provided by an embodiment of the present disclosure, the body includes an upper substrate and a lower substrate; a groove is formed on a surface of the lower substrate; and the upper substrate is packaged on the lower substrate so as to encircle the groove into the culture chamber, the first channel and the second channel.

For example, in the micro-fluidic chip provided by an embodiment of the present disclosure, an inlet and an outlet of the first channel and an inlet of the second channel are all disposed in the upper substrate.

For example, in the micro-fluidic chip provided by an embodiment of the present disclosure, the body is also provided with a third channel and a fourth channel which are arranged opposite to each other; an end of the third channel and an end of the fourth channel are respectively communicated with the culture chamber; the end of the third channel communicated with the culture chamber is arranged opposite to the end of the fourth channel communicated with the culture chamber; the third channel is configured to inject a culture fluid into the culture chamber; and the fourth channel is configured to allow the culture fluid to flow out of the culture chamber.

For example, in the micro-fluidic chip provided by an embodiment of the present disclosure, the body is also provided with a fifth channel; an end of the fifth channel is communicated with the culture chamber; and the fifth channel is configured to introduce gas into the culture chamber.

For example, in the micro-fluidic chip provided by an embodiment of the present disclosure, the body is also provided with a ventilation chamber and an intake passage and an exhaust passage communicated with the ventilation chamber; the intake passage and the exhaust passage are arranged opposite to each other; and an end of the fifth channel away from the culture chamber is communicated with the ventilation chamber, so that an airflow in the ventilation chamber, flowing through the intake passage to the exhaust passage, can flow into the culture chamber through the fifth channel.

For example, in the micro-fluidic chip provided by an embodiment of the present disclosure, an end of the intake passage communicated with the ventilation chamber is arranged opposite to an end of the exhaust passage communicated with the ventilation chamber; and an angle between a connecting line between the end of the intake passage communicated with the ventilation chamber and the end of the exhaust passage communicated with the ventilation chamber and an extension direction of the fifth channel is 75°-90°.

For example, in the micro-fluidic chip provided by an embodiment of the present disclosure, the body is also provided with a sixth channel; an end of the sixth channel is communicated with the ventilation chamber; and the sixth channel is configured to detect a gas in the ventilation chamber.

At least one embodiment of the present disclosure provides a method for separating cells to be cultured employing the micro-fluidic chip according to any one of the above, the method for separating cells to be cultured including: injecting a suspension liquid containing the cells to be cultured into the first channel; and upon a single one of the cells to be cultured being moved to the intersection, injecting a culture fluid into the culture chamber through the second channel, so as to bring the single one of the cells to be cultured disposed at the intersection into the culture chamber.

For example, in the method for separating the cells to be cultured provided by an embodiment of the present disclosure, after injecting the culture fluid into the culture chamber through the second channel so as to bring the single one of the cells to be cultured disposed at the intersection into the culture chamber, the method further includes: injecting a washing fluid into the first channel to wash the first channel.

For example, in the method for separating the cells to be cultured provided by an embodiment of the present disclosure, the cells to be cultured are stein cells.

At least one embodiment of the present disclosure provides a method for manufacturing the micro-fluidic chip according to claim 1, including: forming a lower substrate provided with a groove on a surface by a photolithography method or a hot pressing method; and packaging an upper substrate on the lower substrate so as to encircle the groove into the culture chamber, the first channel and the second channel.

### BRIEF DESCRIPTION OF THE DRAWINGS

Hereinafter, the drawings accompanying embodiments of the present disclosure are simply introduced in order to more clearly explain technical solution(s) of the embodiments of the present disclosure. Obviously, the described drawings below are merely related to some of the embodiments of the present disclosure without constituting any limitation thereto.

FIG. 1 is a schematic structural view of a micro-fluidic chip provided by an embodiment of the present disclosure;

FIG. 2 is a schematic structural view of another micro-fluidic chip provided by an embodiment of the present disclosure;

FIG. 3 is a flowchart of a method for separating cells to be cultured provided by an embodiment of the present disclosure;

FIG. 4 is a flowchart of a method for manufacturing a micro-fluidic chip provided by an embodiment of the present disclosure; and

FIG. 5 is a flowchart of another method for manufacturing a micro-fluidic chip provided by an embodiment of the present disclosure.

### DETAILED DESCRIPTION

In order to make objects, technical details and advantages of the embodiments of the disclosure apparent, technical solutions according to the embodiments of the present disclosure will be described clearly and completely as below in conjunction with the accompanying drawings of embodiments of the present disclosure. Apparently, the described embodiments are only a part of but not all of exemplary embodiments of the present disclosure. Based on the described embodiments of the present disclosure, various other embodiments can be obtained by those of ordinary skill in the art without creative labor and those embodiments shall fall into the protection scope of the present disclosure.

Unless otherwise defined, the technical terminology or scientific terminology used herein should have the same meaning as commonly understood by one of ordinary skill in the art to which this disclosure belongs. The terms “first,” “second,” etc., which are used in the present disclosure, are not intended to indicate any sequence, amount or importance, but distinguish various components. Also, the terms “include,” “including,” “include,” “including,” etc., are intended to specify that the elements or the objects stated before these terms encompass the elements or the objects and equivalents thereof listed after these terms, but do not preclude the other elements or objects. The terms “connection”, “connected” and the like are not limited to physical or mechanical connection but may include electrical connection, either directly or indirectly.

Detailed description on the known functions and the known components are omitted in the present disclosure for clear and concise description of the embodiments of the present disclosure.

In the study, the inventor(s) of the present application notices that stem cell culture is usually carried out on a conventional culture dish, and it is difficult to separate and inject a single cell. In addition, the conventional stein cell culture is to directly inject oxygen into the culture dish, which is very easy to cause cell fluid disturbance, thus affecting the cultivation effect.

In view of this, embodiments of the present disclosure provide a micro-fluidic chip, a method for separating cells to be cultured, and a method for manufacturing the micro-fluidic chip. The micro-fluidic chip includes a body, a culture chamber, a first channel and a second channel, the culture chamber, the first channel and the second channel are located in the body. The first channel and the second channel are intersected to form an intersection; the first channel is configured to transport a suspension liquid containing cells to be cultured; and an end of the second channel is communicated with the culture chamber and configured to inject a culture fluid into the culture chamber so as to bring a cell to be cultured disposed at the intersection into the culture chamber. Thus, the micro-fluidic chip can transport a single cell to be cultured through the first channel and the second channel which are intersected.

Hereinafter, the micro-fluidic chip, the method for separating cells to be cultured and the method for manufacturing the micro-fluidic chip provided by the embodiments of the present disclosure will be described in detail with reference to the accompanying drawings.

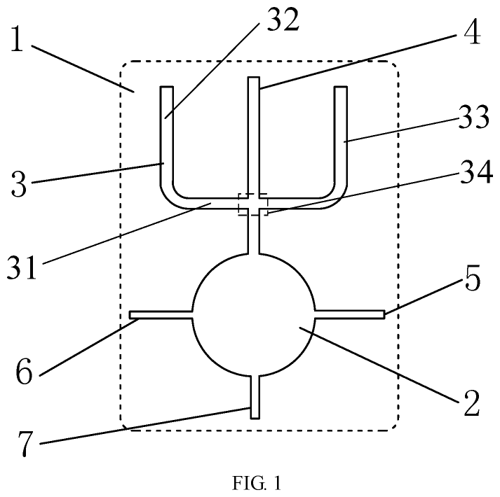

FIG. 1 is a schematic structural view of a micro-fluidic chip provided by an embodiment of the present disclosure. As shown in FIG. 1, the micro-fluidic chip provided by an embodiment of the present disclosure includes a body 1, a culture chamber 2, a first channel 3 and a second channel 4; the culture chamber 2, the first channel 3 and the second channel 4 are located in the body 1. The first channel 3 and the second channel 4 are intersected to form an intersection; the first channel 3 is configured to transport a suspension liquid containing cells to be cultured 12; and an end of the second channel 4 is communicated with the culture chamber 2 and configured to inject a culture fluid into the culture chamber 2 so as to bring a cell to be cultured disposed at the intersection into the culture chamber 2.

In the process of culturing the cell to be cultured 12 by adoption of the micro-fluidic chip provided by the embodiment of the present disclosure, two ends of the first channel 3 are respectively communicated with an injection port and an extraction port of an external cell injection device, and the cell injection device injects the suspension liquid containing the cells to be cultured 12 into an end of the first channel 3 and extracts the suspension liquid from the other end of the first channel 3, so as to guide the suspension liquid to flow along the first channel 3. An end of the second channel 4 away from the culture chamber 2 is connected with an external culture fluid injection device. Upon only one cell to be cultured 12 being located at the intersection, for example, it is observed through a microscope that upon one of the cells to be cultured 12 being just located at the intersection, the culture fluid is injected into the culture chamber 2 through the second channel 4 and can bring a single cell to be cultured 12 disposed at the intersection into the culture chamber 2, so as to achieve the objective of separating the single cell to be cultured 12 and injecting the cell into the culture chamber 2. Thus, the operation is simple, convenient and fast.

In some examples, a cross-sectional dimension of the first channel 3 and the second channel 4 is configured to only allow a single one of the cells to be cultured 12 to pass through, and the second channel 4 is configured to inject the culture fluid into the culture chamber 2 so as to bring a single cell to be cultured 12 disposed at the intersection into the culture chamber 2.

In some examples, the cells to be cultured are stern cells. The first channel 3 and the second channel 4 can only allow a single stem cell 12 to pass through; the first channel 3 is configured to transport a suspension liquid containing the stem cell 12; and the second channel 4 is configured to inject the culture fluid into the culture chamber 2 so as to bring the single stem cell 12 disposed at the intersection into the culture chamber 2. For example, the culture chamber 2 may be a circular culture chamber 2, namely a planar shape of the culture chamber 2 is roughly circular. Of course, the embodiments of the present disclosure include but are not limited thereto.

In the process of culturing the stern cell 12 by adoption of the above micro-fluidic chip, two ends of the first channel 3 can be respectively communicated with an injection port and an extraction port of an external cell injection device, and the cell injection device injects the suspension liquid containing the stein cell 12 into an end of the first channel 3 and extracts the suspension liquid from the other end of the first channel 3, so as to guide the suspension liquid to flow along the first channel 3. An end of the second channel 4 away from the culture chamber 2 is connected with an external culture fluid injection device. Because the first channel 3 and the second channel 4 can only accommodate a single cell to pass through, the stein cells 12 in the suspension liquid are sequentially arranged along the longitudinal direction of the first channel 3 and run through the intersection in sequence. Upon it being observed through, for example, a microscope that a certain stem cell 12 is just located at the intersection, the culture fluid is injected into the culture chamber 2 through the second channel 4 and can bring a single stem cell 12 disposed at the intersection into the culture chamber 2, so as to achieve the objective of separating the single stern cell 12 and injecting the cell into the culture chamber 2. Thus, the operation is simple, convenient and fast.

For example, the diameter of the stem cell 12 is usually tens of microns to 200 microns. In order to allow the first channel 3 and the second channel 4 to only accommodate a single cell to pass through, upon the first channel 3 and the fourth channel 4 being channels with a circular section, the diameter of the first channel 3 and the second channel 4 may be set to be 200 to 250 microns; and upon the first channel 3 and the second channel 4 being channels of which the section is a rectangle, both the long edge and the short edge of the rectangle are set to be 200 to 250 microns.

In some examples, as shown in FIG. 1, an end of the second channel 4 away from the culture chamber 2, the intersection, and an end of the second channel 4 communicated with the culture chamber 2 are arranged in the same straight line.

In some examples, as shown in FIG. 1, the first channel 3 includes a first straight line part 31; the first straight line part 31 and the second channel 4 are intersected to form the intersection; and an included angle between the first straight line part 31 and the second channel 4 is 75°-90°.

For example, as shown in FIG. 1, the first channel 3 further includes a second straight line part 32 and a third straight line part 33. An end of the second straight line part 32 is connected with an inlet of the first channel 3, and the other end is connected with the first straight line part 31; and an end of the third straight line part 33 is connected with the inlet of the first channel 3, and the other end is connected with the first straight line part 31.

For example, as shown in FIG. 1, a reversing valve 34 may be disposed at the intersection of the first channel 3 and the second channel 4 and provided with a valve cavity for accommodating a single one of the cells to be cultured 12 (for example, a single stem cell), and has two working states: in the first working state, the first channel 3 is switched on and the second channel 4 is switched off; and in the second working state, the second channel 4 is switched on and the first channel 3 is switched off. In the process of injecting the suspension liquid containing the stem cell 12 into the first channel 3, the reversing valve 34 can be switched into the first working state, so that the first channel 3 is switched on and the second channel 4 is switched off, thereby avoiding a plurality of stem cells 12 from flowing into the second channel 4 through the intersection. Upon it being observed that a certain stem cell 12 is just located at the intersection, the reversing valve is switched into the second working state, so that the second channel 4 is switched on and the first channel 3 is switched off; and the culture fluid is injected into the culture chamber through the second channel 4 and then can bring a single stern cell 12 disposed in the valve cavity into the culture chamber 2, thereby avoiding the fluid stream from bringing the plurality of stem cells 12 into the culture chamber 2.

In some examples, the body 1 is also provided with a third channel 5 and a fourth channel 6 which are arranged opposite to each other; both opposite ends of the third channel 5 and the fourth channel 6 are communicated with the culture chamber 2; an end of the third channel 5 and an end of the fourth channel 6 are respectively communicated with the culture chamber 2; the end of the third channel 5 communicated with the culture chamber 2 is arranged opposite to the end of the fourth channel 6 communicated with the culture chamber 2; the third channel 5 is configured to inject the culture fluid into the culture chamber 2; and the fourth channel 6 is configured to allow the culture fluid to flow out of the culture chamber 2. The third channel 5 and the fourth channel 6 can be adopted to realize the supply and replacement of the culture fluid during the cultivation process, so as to provide adequate nutrients for the stem cells 12.

In some examples, the body 1 is also provided with a fifth channel 7 of which an end is communicated with the culture chamber 2. During the cultivation process of the stem cells 12, oxygen or air, for example, may be introduced into the culture chamber 2 through the fifth channel 7, so as to ensure that the stein cells 12 undergo cell respiration in a sufficient oxygen environment, thereby ensuring the normal growth and differentiation of the stem cells 12.

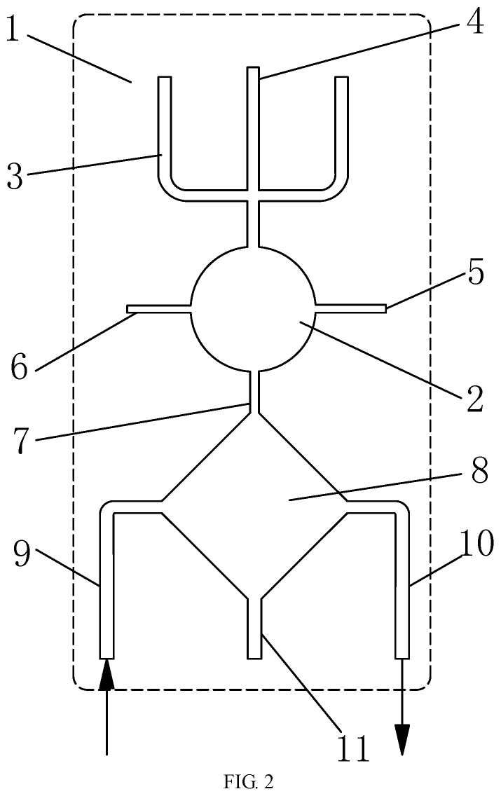

FIG. 2 is a schematic structural view of another micro-fluidic chip provided by one embodiment of the present disclosure. As shown in FIG. 2, the difference between the micro-fluidic chip and the micro-fluidic chip as shown in FIG. 1 is mainly that: the body 1 is also provided with a ventilation chamber 8 and an intake passage 9 and an exhaust passage 10 communicated with the ventilation chamber 8; the intake passage 9 and the exhaust passage 10 are oppositely arranged; an end of the fifth channel 7 away from the culture chamber 2 is communicated with the ventilation chamber 8, so that an airflow in the ventilation chamber 8, flowing through the intake passage 9 to the exhaust passage 10, can flow into the culture chamber 2 through the fifth channel 7; and the fifth channel 7 is disposed between the intake passage 9 and the exhaust passage 10.

In the micro-fluidic chip provided by the embodiment of the present disclosure, an end of the intake passage 9 and an end of the exhaust passage 10 away from the ventilation chamber 8 are connected with an external ventilation device; gas is supplied into the ventilation chamber 8 through the intake passage 9 and extracted from the ventilation chamber 8 through the exhaust passage 10, so as to guide the airflow in the ventilation chamber 8 to flow to the exhaust passage 10 from the intake passage 9; the end of the fifth channel 7 communicated with the ventilation chamber 8 is disposed on a side of the airflow flowing direction; and in the airflow flowing process, a small amount of airflow can flow into the culture chamber 2 through the fifth channel 7, so as to avoid the problem that the disturbance of the culture fluid in the culture chamber 2 can be easily caused upon gas being directly supplied into the culture chamber 2 through the fifth channel 7, and ensure that the stem cell 12 is cultured in a stable environment.

In some examples, as shown in FIG. 2, the end of the intake passage 9 communicated with the ventilation chamber 8 is arranged opposite to the end of the exhaust passage 10 communicated with the ventilation chamber 8; and an included angle between a connecting line between the end of the intake passage 9 communicated with the ventilation chamber 8 and the end of the exhaust passage 10 communicated with the ventilation chamber 8 and an extension direction of the fifth channel 7 is 75°-90°, so as to further reduce the impact of the airflow between the intake passage 9 and the exhaust passage 10 on the culture chamber 2.

For example, the connecting line between the end of the intake passage 9 communicated with the ventilation chamber 8 and the end of the exhaust passage 10 communicated with the ventilation chamber 8 is perpendicular to the extension direction of the fifth channel 7.

In some examples, as shown in FIG. 2, the body 1 is also provided with a sixth channel 11; an end of the sixth channel 11 is communicated with the ventilation chamber 8; and the sixth channel 11 is configured to detect the gas in the ventilation chamber 8, so as to regulate and control the gas environment in the ventilation chamber 8 in real time and ensure that the stem cells 12 in the culture chamber 2 undergo cell respiration in an adequate oxygen environment.

In some embodiments, as shown in FIG. 2, the ventilation chamber 8 is a rhombic ventilation chamber 8; the intake passage 9 and the exhaust passage 10 are respectively connected to one pair of opposite vertexes of the rhombic ventilation chamber 8; and the fifth channel 7 and the sixth channel 11 are respectively connected to another pair of opposite vertexes of the rhombic ventilation chamber 8. By arrangement of the ventilation chamber 8 to be rhombic, smooth and stable airflow can be formed in the fifth channel 7 and supplied into the culture chamber 2. Of course, the ventilation chamber 8 is not limited to the rhombic ventilation chamber 8 and may also adopt other shapes.

In some embodiments, the body 1 may include a lower substrate 16 and an upper substrate 17; a groove is formed on a surface of the lower substrate 16; and the upper substrate 17 is packaged on the lower substrate 16 to encircle the groove into the cell culture camber 2, the ventilation chamber 8, the first channel 3, the second channel 4, the third channel 5, the fourth channel 6, the fifth channel 7, the sixth channel 11, the intake passage 9 and the exhaust passage 10.

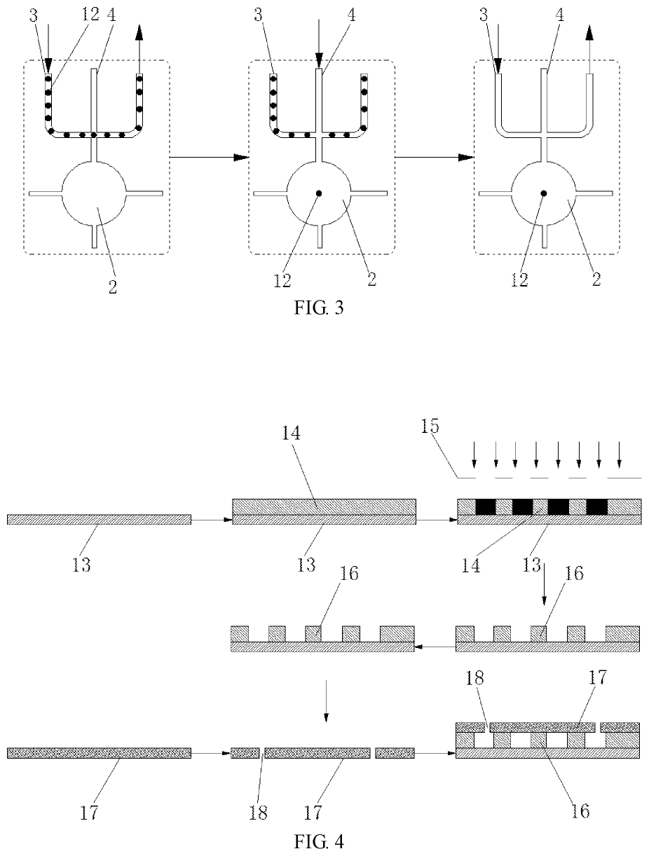

FIG. 3 is a flowchart of a method for separating cells to be cultured provided by one embodiment of the present disclosure. As shown in FIG. 3, the method for separating the cells to be cultured employing the above micro-fluidic chip, provided by one embodiment of the present disclosure, includes:

S110: injecting a suspension liquid containing cells to be cultured 12 into the first channel 3.

For example, the reversing valve 34 can be switched into the first working state, so that the first channel 3 is switched on and the second channel 4 is switched off; two ends of the first channel 3 are respectively communicated with an injection port and an extraction port of an external cell injection device; and the cell injection device injects a suspension liquid containing stem cells 12 from an end of the first channel 3 and extracts the suspension liquid from the other end of the first channel 3, so as to guide the suspension liquid to flow along the first channel 3.

S120: upon a single one of the cells to be cultured 12 being moved to the intersection, injecting a culture fluid into the culture chamber 2 through the second channel 4 so as to bring the single one of the cells to be cultured 12 disposed at the intersection into the culture chamber 2.

For example, an end of the second channel 4 away from the culture chamber 2 is connected with an external culture fluid injection device; after observing the movement of cells in the first channel 3 by, for example, a microscope, upon a single one of the cells to be cultured 12 being moved to the intersection, the reserving valve can be switched into the second working state, so that the second channel is switched on and the first channel is switched off; and subsequently, the culture fluid is injected into the culture chamber 2 through the second channel 4; and the culture fluid can bring the single cell to be cultured 12 disposed at the intersection into the culture chamber 2 while flowing to the culture chamber 2, so as to achieve the objective of separating and injecting the single cell to be cultured 12. Thus, the operation is simple, convenient and fast.

In some examples, after injecting the culture fluid into the culture chamber 2 through the second channel 4 so as to bring the stein cell 12 disposed at the intersection into the culture chamber 2, the method further includes:

S130: injecting a washing fluid into the first channel 3 to wash the first channel 3, so as to wash out the suspension liquid containing the cell to be cultured 12 (for example, the stein cell) from the first channel 3, thereby avoiding the waste of the cell to be cultured 12 and also avoiding the case that the remaining cells to be cultured 12 in the first channel 3 enter the culture chamber 2 due to misoperation to affect the culture experiment of the single cell to be cultured 12.

FIG. 4 is a flowchart of a method for manufacturing a micro-fluidic chip provided by one embodiment of the present disclosure. As shown in FIG. 4, the method for manufacturing the micro-fluidic chip provided by one embodiment of the present disclosure includes the following steps:

S210: forming a lower substrate 16 provided with a groove on a surface by a photolithography method or a hot pressing method.

S220: packaging an upper substrate 17 on the lower substrate 16, so as to encircle the groove into cavity and channel structures such as a culture chamber 2, a ventilation chamber 8, a first channel 3, a second channel 4, a third channel 5, a fourth channel 6, a fifth channel 7, a sixth channel 11, an intake passage 9 and an exhaust passage 10.

The adoption of the above manufacturing method to manufacture the micro-fluidic chip can accurately control the size of the micro-fluidic chip and improve the manufacturing efficiency of the micro-fluidic chip.

In some embodiments, packaging the upper substrate 17 on the lower substrate 16 includes:

S221: forming a plurality of through holes 18 respectively communicated with an external device on the upper substrate 17. Optionally, the through holes 18 are formed by, for example, a laser etching process, so both the opening efficiency and the opening accuracy are high.

S222: packaging the upper substrate 17 on the lower substrate 16, so as to encircle the groove into cavity and channel structures such as the culture chamber 2, the ventilation chamber 8, the first channel 3, the second channel 4, the third channel 5, the fourth channel 6, the fifth channel 7, the sixth channel 11, the intake passage 9 and the exhaust passage 10, and allow the plurality of through holes to be respectively communicated with an inlet end and an outlet end of the first channel 3, an inlet end of the second channel 4, an inlet end of the third channel 5, an outlet end of the fourth channel 6, an end of the sixth channel 11 away from the ventilation chamber 8, an inlet end of the intake passage 9, and an outlet end of the exhaust passage 10, so as to respectively form an inlet and an outlet of the first channel 3, an inlet of the second channel 4, an inlet of the third channel 5, an outlet of the fourth channel 6, a sampling port of the sixth channel 11, an inlet of the intake passage 9, and an outlet of the exhaust passage 10.

For example, a cell injection device may be connected with the first channel 3 through the through hole 18 communicated with the first channel 3; a culture fluid injection device may be connected with the second channel 4 through the through hole 18 communicated with the second channel 4, and may be respectively connected with the third channel 5 and the fourth channel 6 through the through holes 18 communicated with the third channel 5 and the fourth channel 6; a gas exchange device may be respectively connected with the intake passage 9 and the exhaust passage 10 through the through holes 18 communicated with the intake passage 9 and the exhaust passage 10; and a gas detection device may be connected with the sixth channel 11 through the through hole 18 communicated with the sixth channel 11.

As shown in FIG. 4, in some embodiments, the step of manufacturing the lower substrate 16 provided with the groove on the surface at least by photolithography specifically includes the following steps:

S211: coating photoresist 14 on a surface of a substrate 13. The substrate 13 may be a glass substrate 13 and may also be a resin substrate 13. The embodiment adopts the glass substrate 13.

S212: forming a mask 15 on the substrate coated with the photoresist 14.

S213: forming the lower substrate 16 provided with the groove on the surface by performing exposure on the substrate 13 provided with the mask 15.

Without being limited to any theory, it is considered that the production of the lower substrate 16 provided with the groove on the surface by the process has the advantages of simple process and high production efficiency.

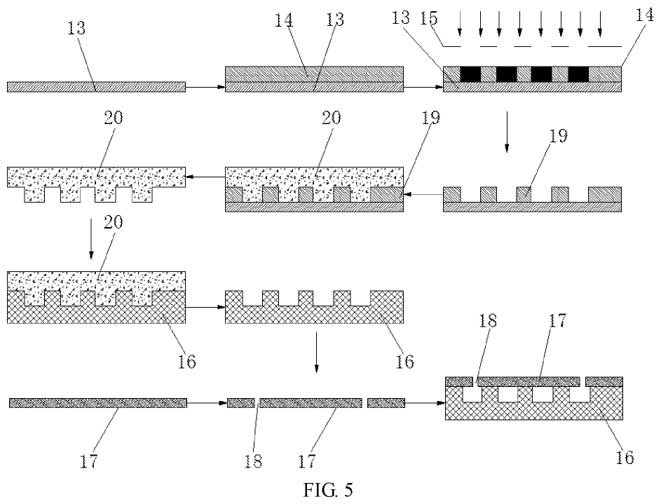

FIG. 5 is a flowchart of another method for manufacturing a micro-fluidic chip provided by an embodiment of the present disclosure. As shown in FIG. 5, the difference between the manufacturing method of the micro-fluidic chip and the above manufacturing method of the micro-fluidic chip is mainly the steps of manufacturing the lower substrate 16 provided with the groove on the surface. In the embodiment, the process of manufacturing the lower substrate 16 provided with the groove on the surface at least by photolithography specifically includes the following steps:

S211′: forming a groove on the surface of the substrate 13 coated with photoresist 14 by photolithography, and taking the groove as a first mould 9.

The step is similar to the step of manufacturing the lower substrate 16 provided with the groove on the surface in the first embodiment, namely includes: coating the photoresist 14 on the surface of the substrate 13; forming a mask 15 on the substrate 13 coated with the photoresist 14; and forming the groove on the surface of the substrate 13 coated with the photoresist 14 by performing exposure on the substrate 13 provided with the mask 15, and taking the groove as the first mould 9.

S212′: forming a second mould 20 by depositing metal on the first mould 19. Optionally, metal may be deposited on the first mould 19 by deposition process such as magnetron sputtering, and the second mould 20 is formed after demoulding.

S213′: forming the lower substrate 16 provided with the groove on the surface by performing hot pressing on the lower substrate 16 by adoption of the second mould 20. The lower substrate 16 may adopt a resin substrate.

Without being limited to any theory, it is considered that the lower substrate 16 provided with the groove on the surface is formed by the process in the embodiment; the second mold 20 can be repeatedly used; and the formed lower substrate 16 has good consistency and high productivity.

The following statements should be noted:

(1) The accompanying drawings involve only the structure(s) in connection with the embodiment(s) of the present disclosure, and other structure(s) can be referred to common design(s).

(2) In case of no conflict, features in one embodiment or in different embodiments can be combined.

The foregoing are merely specific embodiments of the present disclosure, but not limitative to the protection scope of the present disclosure. Within the technical scope disclosed by the present disclosure, any alternations or replacements which can be readily envisaged by one skilled in the art shall be within the protection scope of the present disclosure. Therefore, the protection scope of the invention shall be defined by the accompanying claims.

What is claimed is:

1. A micro-fluidic chip, comprising a body and a culture chamber, a first channel and a second channel, the culture chamber, the first channel, and the second channel being located in the body,
wherein the first channel and the second channel are intersected to form an intersection; the first channel is configured to transport a suspension liquid containing cells to be cultured; and an end of the second channel is communicated with the culture chamber and configured to inject a culture fluid into the culture chamber so as to bring a cell to be cultured disposed at the intersection into the culture chamber.

2. The micro-fluidic chip according to claim 1, wherein a cross-sectional dimension of the first channel and the second channel is configured to only allow a single one of the cells to be cultured to pass through; and the second channel is configured to inject the culture fluid into the culture chamber so as to bring a single cell to be cultured disposed at the intersection into the culture chamber.

3. The micro-fluidic chip according to claim 2, wherein the cells to be cultured are stem cells.

4. The micro-fluidic chip according to claim 1, wherein an end of the second channel away from the culture chamber, the intersection, and the end of the second channel communicated with the culture chamber are arranged in a same straight line.

5. The micro-fluidic chip according to claim 4, wherein the first channel includes a first straight line part; the first straight line part and the second channel are intersected to form the intersection; and an included angle between the first straight line part and the second channel is 75°-90°.

6. The micro-fluidic chip according to claim 1, wherein a reversing valve is disposed at the intersection and provided with a valve cavity for accommodating a single one of the cells to be cultured, and has two working states: in a first working state, the first channel is switched on and the second channel is switched off; and in a second working state, the second channel is switched on and the first channel is switched off.

7. The micro-fluidic chip according to claim 6, wherein the cells to be cultured are stem cells.

8. The micro-fluidic chip according to claim 1, wherein the body includes an upper substrate and a lower substrate; a groove is formed on a surface of the lower substrate; and the upper substrate is packaged on the lower substrate so as to encircle the groove into the culture chamber, the first channel and the second channel.

9. The micro-fluidic chip according to claim 8, wherein an inlet and an outlet of the first channel and an inlet of the second channel are all disposed in the upper substrate.

10. The micro-fluidic chip according to claim 1, wherein the body is also provided with a third channel and a fourth channel which are arranged opposite to each other; an end of the third channel and an end of the fourth channel are respectively communicated with the culture chamber; the end of the third channel communicated with the culture chamber is arranged opposite to the end of the fourth channel communicated with the culture chamber; the third channel is configured to inject a culture fluid into the culture chamber; and the fourth channel is configured to allow the culture fluid to flow out of the culture chamber.

11. The micro-fluidic chip according to claim 1, wherein the body is also provided with a fifth channel; an end of the fifth channel is communicated with the culture chamber; and the fifth channel is configured to introduce gas into the culture chamber.

12. The micro-fluidic chip according to claim 11, wherein the body is also provided with a ventilation chamber and an intake passage and an exhaust passage communicated with the ventilation chamber; the intake passage and the exhaust passage are arranged opposite to each other; and an end of the fifth channel away from the culture chamber is communicated with the ventilation chamber, so that an airflow in the ventilation chamber, flowing through the intake passage to the exhaust passage, can flow into the culture chamber through the fifth channel.

13. The micro-fluidic chip according to claim 12, wherein an end of the intake passage communicated with the ventilation chamber is arranged opposite to an end of the exhaust passage communicated with the ventilation chamber; and an angle between a connecting line between the end of the intake passage communicated with the ventilation chamber and the end of the exhaust passage communicated with the ventilation chamber and an extension direction of the fifth channel is 75°-90°.

14. The micro-fluidic chip according to claim 12, wherein the body is also provided with a sixth channel; an end of the sixth channel is communicated with the ventilation chamber; and the sixth channel is configured to detect a gas in the ventilation chamber.

15. A method for separating cells to be cultured employing the micro-fluidic chip according to claim 1, comprising:
injecting a suspension liquid containing the cells to be cultured into the first channel; and
upon a single one of the cells to be cultured being moved to the intersection, injecting a culture fluid into the culture chamber through the second channel, so as to bring the single one of the cells to be cultured disposed at the intersection into the culture chamber.

16. The method for separating the cells to be cultured according to claim 15, wherein after injecting the culture fluid into the culture chamber through the second channel so as to bring the single one of the cells to be cultured disposed at the intersection into the culture chamber, the method further comprises:
injecting a washing fluid into the first channel to wash the first channel.

17. The method for separating the cells to be cultured according to claim 15, wherein the cells to be cultured are stem cells.

18. A method for manufacturing the micro-fluidic chip according to claim 1, comprising:
forming a lower substrate provided with a groove on a surface by a photolithography method or a hot pressing method; and
packaging an upper substrate on the lower substrate so as to encircle the groove into the culture chamber, the first channel and the second channel.
